(19) United States
(12) Patent Application Publication
(10) Pub. No.: US 2022/0293239 A1
(43) Pub. Date: Sep. 15, 2022

(54) RECIPE OUTPUT METHOD AND RECIPE OUTPUT SYSTEM

(71) Applicant: Panasonic Intellectual Property Management Co., Ltd., Osaka (JP)

(72) Inventors: Junko NAKAJIMA, Osaka (JP); Masashi ISHIMARU, Osaka (JP); Jakusei KIYOSAKI, Hyogo (JP); Yoshihiro MATSUMURA, Osaka (JP)

(21) Appl. No.: 17/632,448

(22) PCT Filed: Aug. 27, 2020
(86) PCT No.: PCT/JP2020/032303

§ 371 Date: Feb. 2, 2022

(30) Foreign Application Priority Data
Sep. 24, 2019 (JP) 2019-173194

(51) Int. Cl. G16H 20/60; A61B 5/00; G10L 25/66

(52) U.S. Cl. CPC G16H 20/60; A61B 5/4205; A61B 5/4803; A61B 5/742; G10L 25/66

(57) ABSTRACT

A recipe output method includes: receiving a selection of a dish from a menu indicating a plurality of dishes; acquiring ability information indicating an eating and swallowing function of a subject determined by evaluation based on a speech spoken by the subject; and outputting a recipe for cooking the dish for which the selection was received, the recipe being adjusted to be suitable for the eating and swallowing function of the subject indicated by the ability information acquired.

### TECHNICAL FIELD

The present invention relates to a recipe output method and a recipe output system that output a recipe.

### BACKGROUND ART

Conventionally, a menu suggesting system is known that suggests, based on a purchase history of food items purchased by a menu (or in other words, dish) recipient, a menu that uses ingredients for constituting (cooking) the menu that match the food items recorded in the purchase history (see Patent Literature (PTL) 1).

### CITATION LIST

### Patent Literature

[PTL 1] Japanese Unexamined Patent Application Publication No. 2004-13331

### SUMMARY OF INVENTION

### Technical Problem

However, some people may have difficulty in eating food cooked based on an ordinary recipe, and even eating the food can be dangerous.

To address this, it is an object of the present invention to provide a recipe output method and the like that output a recipe for cooking food suitable for a subject.

### Solution to Problem

A recipe output method according to one aspect of the present invention includes: receiving a selection of a dish from a menu indicating a plurality of dishes; acquiring ability information indicating an eating and swallowing function of a subject determined by evaluation based on a speech spoken by the subject; and outputting a recipe for cooking the dish for which the selection was received, the recipe being adjusted to be suitable for the eating and swallowing function of the subject indicated by the ability information acquired.

Also, recipe output system according to one aspect of the present invention includes: a receiver that receives a selection of a dish from a menu indicating a plurality of dishes; an acquirer that acquires ability information indicating an eating and swallowing function of a subject determined by evaluation based on a speech spoken by the subject; and an outputter that outputs a recipe for cooking the dish for which the selection was received, the recipe being adjusted to be suitable for the eating and swallowing function of the subject indicated by the ability information acquired.

### Advantageous Effects of Invention

With the recipe output method and the like of the present invention, it is possible to output a recipe for cooking food suitable for a subject.

### BRIEF DESCRIPTION OF DRAWINGS

FIG. 1 is a schematic diagram showing a configuration of a recipe output system according to an embodiment.

FIG. 2 is a block diagram showing a characteristic functional configuration of the recipe output system according to the embodiment.

FIG. 3A is a diagram showing an example of speech data of a speech spoken by a subject.

FIG. 3B is a frequency spectrum diagram illustrating formant frequencies.

FIG. 3C is a diagram showing an example of variation of formant frequencies with time.

FIG. 3D is a diagram showing specific examples of eating and swallowing functions in a preparation stage, a mouth cavity stage, and a pharynx stage, and symptoms that occur when the eating and swallowing functions are impaired.

FIG. 4 is a diagram showing an example of ability information.

FIG. 5 is a flowchart illustrating a processing procedure for outputting a recipe according to the embodiment.

FIG. 6 is a first modification table in which eating and swallowing functions and modifications to be made to an output recipe are associated with each other.

FIG. 7 is a second modification table in which eating and swallowing functions and modifications to be made to an output recipe are associated with each other.

FIG. 8A is a first diagram showing an example of a recipe output according to an example.

FIG. 8B is a first diagram showing an example of a recipe output according to a comparative example.

FIG. 9A is a second diagram showing an example of the recipe output according to the example.

FIG. 9B is a second diagram showing an example of the recipe output according to the comparative example.

### DESCRIPTION OF EMBODIMENTS

Hereinafter, an embodiment will be described with reference to the drawings. The embodiment described below shows a generic or specific example of the present invention. The numerical values, shapes, materials, structural elements, the arrangement and connection of the structural elements, steps, the order of the steps, and the like shown in the following embodiment are merely examples, and therefore are not intended to limit the scope of the present invention. Also, among the structural elements described in the following embodiment, structural elements not recited in any one of the independent claims are described as arbitrary structural elements.

The diagrams are schematic representations, and thus are not necessarily true to scale. Also, in the diagrams, structural elements that are substantially the same are given the same reference numerals, and a redundant description may be omitted or simplified.

### EMBODIMENT

### [Eating and Swallowing Functions]

The present invention relates to outputting a recipe adjusted to be suitable for an eating and swallowing function of a subject determined by evaluation. First, eating and swallowing functions will be described.

Eating and swallowing functions are human body functions needed to achieve a sequence of processes from recognizing food, moving the food into the mouth, and ingesting the food into the stomach. The eating and swallowing functions include five stages: an initial stage; a preparation stage; a mouth cavity stage; a pharynx stage; and an esophagus stage.

In the initial stage (also referred to as “recognition stage”) of eating and swallowing, the shape, the hardness, the temperature, and the like of food are determined. The eating and swallowing function in the initial stage includes, for example, a visual recognition function, and the like. In the initial stage, the properties and the state of food are recognized, and preparations needed for eating are made such as the eating manner, the salivation, and the posture.

In the preparation stage (also referred to as “mastication stage”) of eating and swallowing, the food taken into the mouth cavity is crushed and ground by the teeth (or in other words, masticated), and the masticated food is mixed with saliva by the tongue to gather the food into a food bolus. The eating and swallowing function in the preparation stage includes: for example, a facial muscle (lip muscles, cheek muscles, and the like) movement function of moving the food into the mouth cavity without spilling the food; a tongue recognition function of recognizing the taste and the hardness of the food; a tongue movement function of pushing the food against the teeth and mixing the finely crushed food with saliva to gather the food into a food bolus; a state of dental occlusion of the teeth for crushing and grinding the food; a cheek movement function of preventing the food from entering between the teeth and the cheeks; a mastication muscle (masseter muscle, temporalis muscle, and the like) movement function (mastication function), the mastication muscles being a general term for muscles used for mastication; a salivation function of forming the finely crushed food to gather the food into a food bolus; and the like. The mastication function is affected by the state of dental occlusion, the mastication muscle movement function, the tongue function, and the like. With the eating and swallowing function in the preparation stage, the physical properties (size, lump, viscosity, and adhesion) of the food bolus are turned such that the food bolus is easily swallowed, and thus the food bolus smoothly moves from the mouth cavity to the stomach through the pharynx.

In the mouth cavity stage of eating and swallowing, the tongue (the tip of the tongue) is raised to move the food bolus from the mouth cavity to the pharynx. The eating and swallowing function in the mouth cavity stage includes: for example, a tongue movement function of moving the food bolus to the pharynx, a soft palate raising function of closing a space between the pharynx and the nasal cavity, and the like.

In the pharynx stage of eating and swallowing, when the food bolus arrives at the pharynx, due to the occurrence of a swallowing reflex, the food bolus is transferred to the esophagus in a short period of time (about one second). Specifically, the soft palate is raised to close the space between the pharynx and the nasal cavity, and the root of the tongue (specifically, the hyoid bone that supports the root of the tongue) and the larynx are raised to allow the food bolus to pass through the pharynx. At this time, the epiglottis flips down to close the entrance of the trachea, and the food bolus is transferred to the esophagus such that accidental aspiration does not occur. The eating and swallowing function in the pharynx stage includes: for example, a pharynx movement function of closing the space between the pharynx and the nasal cavity (specifically, a movement function of raising the soft palate); a tongue (specifically, the root of the tongue) movement function of transferring the food bolus to the pharynx; a larynx movement function of transferring the food bolus from the pharynx to the esophagus, or when the food bolus flows into the pharynx, closing the glottis to close the trachea and closing the entrance of the trachea as a result of the epiglottis hanging down to the entrance of the trachea; and the like.

In the esophagus stage of eating and swallowing, peristalsis of the esophagus wall is induced, and the food bolus is transferred from the esophagus into the stomach. The eating and swallowing function in the esophagus stage includes, for example, an esophagus peristalsis function of moving the food bolus to the stomach, and the like.

According to the present invention, it is possible to output a recipe for cooking food suitable for the eating and swallowing function of the subject based on the eating and swallowing function of the subject determined by evaluating a speech spoken by the subject.

Specific features are observed in speeches spoken by evaluatees with impaired eating and swallowing functions. Accordingly, by calculating the features as feature values, the eating and swallowing function of an evaluatee can be evaluated. Hereinafter, the evaluation of the eating and swallowing functions in the preparation stage, the mouth cavity stage, and the pharynx stage will be described. The present invention is implemented by a recipe output method and a recipe output system for carrying out the recipe output method. Hereinafter, the recipe output method will be described by referring to the recipe output system.

### [Configuration of Recipe Output System]

A configuration of a recipe output system according to an embodiment will be described.

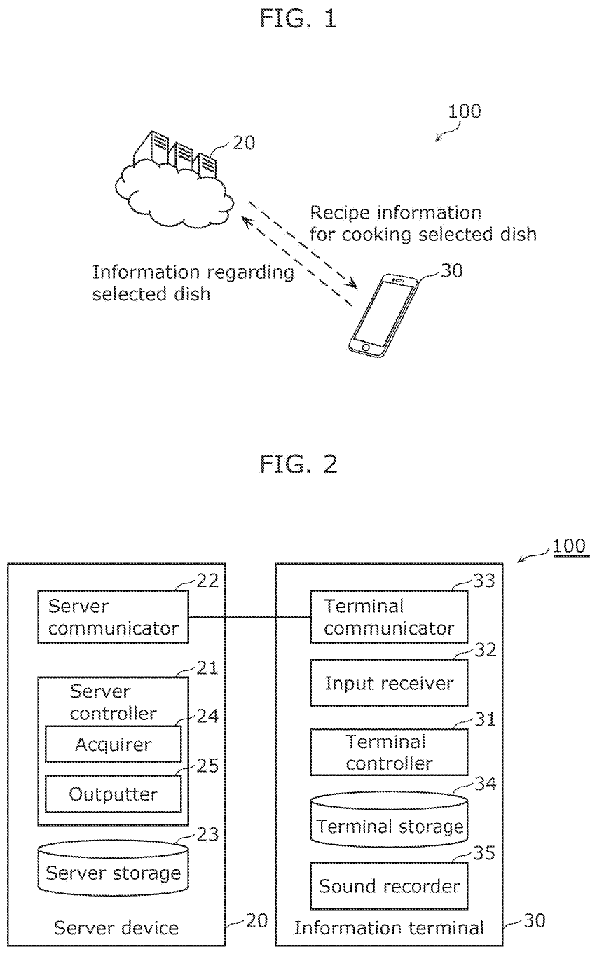

FIG. 1 is a schematic diagram showing a configuration of a recipe output system according to the embodiment.

Recipe output system 100 is a system that outputs a recipe for cooking food based on the eating and swallowing function of a subject determined by analyzing and evaluating a speech spoken by the subject. As shown in FIG. 1, recipe output system 100 includes server device 20 and information terminal 30.

Server device 20 is a device that receives dish information regarding a dish selected by the subject according to the eating and swallowing function, outputs a recipe for cooking the dish indicated by the dish information, and transmits the recipe to information terminal 30 as recipe information. Server device 20 is also a device that receives speech data indicating a speech spoken by the subject by using information terminal 30, and evaluates the eating and swallowing function of the subject based on the received speech data. Recipe output system 100 may also include an evaluation device for evaluating the eating and swallowing function of the subject, separately from server device 20. Recipe output system 100 does not necessarily need to include the evaluation device for evaluating the eating and swallowing function of the subject as long as recipe output system 100 is configured to acquire the eating and swallowing function of the subject that has been determined by evaluation in advance.

Information terminal 30 is a device that receives a selection of a dish selected by the subject, transmits the recipe to server device 20 as dish information, and presents recipe information received as a result. Also, information terminal 30 includes sound recorder 35 (see FIG. 2 described later) that records, in a non-contact manner, a speech obtained as a result of the subject speaking a predetermined syllable or a predetermined sentence, and transmits speech data indicating the recorded speech to server device 20.

For example, information terminal 30 may be a smart phone, a tablet terminal, or the like that includes a microphone that is an example of sound recorder 35. Information terminal 30 is not limited to a smart phone or a tablet terminal, and may be, for example, a notebook PC or the like. Also, recipe output system 100 may include, instead of information terminal 30, a sound recording device such as a microphone, as a sound recorder. However, recipe output system 100 does not necessarily need to include a sound recorder as described above. This is because, as long as ability information indicating the eating and swallowing function of the subject output in advance based on the speech spoken by the subject can be acquired, it is unnecessary to perform an additional evaluation of the eating and swallowing function of the subject.

Also, information terminal 30 may include a display device, such as a display, that displays images and the like based on image data output from server device 20. The display device does not necessarily need to be included in information terminal 30, and may be a separate monitor device that includes a liquid crystal panel, an organic EL panel, or the like.

Server device 20 and information terminal 30 may be connected in a wired or wireless manner, or may be connected via a broad band network such as the Internet. That is, the subject can select a dish and obtain a recipe for cooking the dish according to the present embodiment as long as the subject holds information terminal 30, such as a smart phone, that is connected to a broad band network that is connected to server device 20.

Server device 20 analyzes the speech of the subject based on the speech data recorded by information terminal 30, evaluates the eating and swallowing function of the subject based on the result of analysis, and outputs ability information as a result of analysis. The functions of server device 20, specifically, the function of outputting a recipe and the function of evaluating the eating and swallowing function of the subject may be implemented by a personal computer, instead of server device 20. Also, the functions of server device 20 of outputting a recipe and evaluating the eating and swallowing function of the subject may be combined into information terminal 30. In this case, recipe output system 100 can be implemented by using information terminal 30 alone.

FIG. 2 is a block diagram showing a characteristic functional configuration of the recipe output system according to the embodiment. Server device 20 includes server controller 21, server communicator 22, and server storage 23.

Server controller 21 includes acquirer 24 and outputter 25.

Acquirer 24 includes a processing unit that acquires the ability information indicating the eating and swallowing function of the subject. Acquirer 24 is also a processing unit that acquires the speech data obtained as a result of information terminal 30 recording a speech spoken by the subject in a non-contact manner. The speech may be a speech obtained as a result of the subject speaking a predetermined syllable or a predetermined sentence, or may be a speech obtained by trimming a speech need to evaluate the eating and swallowing function of the subject from among speeches spoken by the subject in ordinary conversations. The processing unit of acquirer 24 is implemented by a processor and a memory that is connected to the processor. The processing unit implements the above-described functions of acquirer 24 as a result of the processor executing a program for performing various processing operations stored in the memory.

Outputter 25 is a processing unit that outputs a recipe for cooking the dish for which the selection was received, the recipe being adjusted to be suitable for the eating and swallowing function of the subject. Outputter 25 is also a processing unit that evaluates the eating and swallowing function of the subject based on the speech spoken by the subject, and outputs ability information as a result of evaluation. The processing unit of outputter 25 is implemented by a processor and a memory that is connected to the processor. The processing unit implements the above-described functions of outputter 25 as a result of the processor executing a program for performing various processing operations stored in the memory.

Server communicator 22 is a communication module for communicably connecting server device 20 and information terminal 30.

Server storage 23 is a storage device for storing information used in server device 20. Server storage 23 is implemented by, for example, a ROM (Read Only Memory), a RAM (Random Access Memory), a semiconductor memory, a HDD (Hard Disk Drive), or the like. Server storage 23 also stores programs that are executed by the processing units, recipe information for cooking food to be output, images that indicate image data that shows a result of evaluation of the eating and swallowing function of the subject used when outputting the result of evaluation, data such as moving images, speeches, and texts, and the like.

Although not shown in the diagrams, server device 20 may include an instruction provider that provides, to the subject, an instruction to pronounce a predetermined syllable or a predetermined sentence. Specifically, the instruction provider acquires image data of an image for providing an instruction to pronounce a predetermined syllable or a predetermined sentence and speech data that have been stored in server storage 23, and outputs the image data and the speech data to information terminal 30.

Information terminal 30 includes terminal controller 31, input receiver 32, terminal communicator 33, terminal storage 34, and sound recorder 35.

Terminal controller 31 includes a processing unit for implementing various functions of information terminal 30. The processing unit of terminal controller 31 is implemented by a processor and a memory that is connected to the processor. The processing unit implements the above-described functions of terminal controller 31 as a result of the processor executing a program for performing various processing operations stored in the memory.

Input receiver 32 is an example of a receiver, and may be a user interface for receiving an operation input to information terminal 30 by the subject. Input receiver 32 is implemented by, for example, an input device, such as a touch panel, that also functions as the display described above.

Terminal communicator 33 is a communication module for communicably connecting server device 20 and information terminal 30.

Terminal storage 34 is a storage device for storing information used in information terminal 30. Terminal storage 34 is implemented by, for example, a ROM, a semiconductor memory, a HDD, or the like.

Sound recorder 35 is a sound recording module such as a microphone that is used to record the speech spoken by the subject and is included in information terminal 30. Sound recorder 35 records the speech spoken by the subject as speech data.

### [Processing Procedure for Evaluating Eating and Swallowing Function]

Next, a specific processing procedure for evaluating the eating and swallowing function performed by server device 20 will be described.

First, a speech that includes a predetermined syllable or a predetermined sentence (a sentence that includes a specific sound) spoken by the subject is recorded. For example, the image data of an image for providing an instruction to the subject acquired by the instruction provider is output to information terminal 30. The image for providing an instruction to the subject is displayed on the display of information terminal 30. The predetermined sentence included in the instruction may be “ki ta ka ra ki ta ka ta ta ta ki ki”, “e wo ka ku ko to ni ki me ta”, “ki ta ka ze to ta i yo u”, “a i u e o”, “pa pa pa pa pa . . . ”, “ta ta ta ta ta . . . ”, “ka ka ka ka ka . . . ”, “ra ra ra ra ra . . . ”, “pa n da no ka ta ta ta ki”, or the like. The pronunciation instruction does not necessarily need to be an instruction to pronounce a predetermined sentence, and may be an instruction to pronounce a predetermined syllable of a single hiragana character such as “ki”, “ta”, “ka”, “ra”, “ze”, or “pa”. Also, the pronunciation instruction may be an instruction to pronounce a meaningless phrase that includes only vowels of two or more syllables such as “e o” or “i e a”. The pronunciation instruction may be an instruction to repeatedly pronounce a meaningless phrase as described above.

Also, the instruction provider may provide the instruction to pronounce a predetermined syllable, a predetermined sentence voice, or the like by using voice, without using the image for providing the instruction, by acquiring voice data for providing the instruction to the subject that has been stored in server storage 23 and outputting the voice data to information terminal 30. Furthermore, the instruction may be provided by using the voice of an evaluator speaking to the subject, without using the image and the voice for providing the instruction, the evaluator being a person (a family member, a doctor, or the like) who wants to evaluate the eating and swallowing function of the subject.

For example, the predetermined syllable may be composed of a consonant and a vowel that follows the consonant. In the case of Japanese, for example, the predetermined syllable may be “ki”, “ta”, “ka”, “ze”, or the like. “ki” is composed of a consonant “k” and a vowel “i” that follows the consonant. “ta” is composed of a consonant “t” and a vowel “a” that follows the consonant. “ka” is composed of a consonant “k” and a vowel “a” that follows the consonant. “ze” is composed of a consonant “z” and a vowel “e” that follows the consonant.

Also, for example, the predetermined sentence may include a syllable part composed of a consonant, a vowel that follows the consonant, and a consonant that follows the vowel. In the case of Japanese, for example, the syllable part may be “kaz” in “kaze”. Specifically, the syllable part is composed of a consonant “k”, a vowel “a” that follows the consonant, and a consonant “z” that follows the vowel.

Also, for example, the predetermined sentence may include a character string that includes successive syllables that include vowels. In the case of Japanese, for example, the character string may be “a i u e o”, or the like.

Also, for example, the predetermined sentence may include a predetermined word. In the case of Japanese, for example, the predetermined word may be “ta i yo u: taiyou (that means the sun in Japanese)”, “ki ta ka ze: kitakaze (that means north wind in Japanese)”, or the like.

Also, for example, the predetermined sentence may include a phrase in which a syllable composed of a consonant and a vowel that follows the consonant is repeated. In the case of Japanese, for example, the phrase may be “pa pa pa pa pa . . . ”, “ta ta ta ta ta . . . ”, “ka ka ka ka ka . . . ”, “ra ra ra ra ra . . . ”, or the like. “pa” is composed of a consonant “p” and a vowel “a” that follows the consonant. “ta” is composed of a consonant “t” and a vowel “a” that follows the consonant. “ka” is composed of a consonant “k” and a vowel “a” that follows the consonant. “ra” is composed of a consonant “r” and a vowel “a” that follows the consonant.

Sound recorder 35 records the speech data of the subject who has received the instruction. For example, the subject speaks a predetermined sentence such as “ki ta ka ra ki ta ka ta ta ta ki ki” toward sound recorder 35 of information terminal 30. Sound recorder 35 records the predetermined sentence or a speech that includes a predetermined syllable spoken by the subject, as speech data.

Next, outputter 25 of server controller 21 calculates feature values from the speech data recorded by sound recorder 35, and evaluates the eating and swallowing function of the subject based on the calculated feature values.

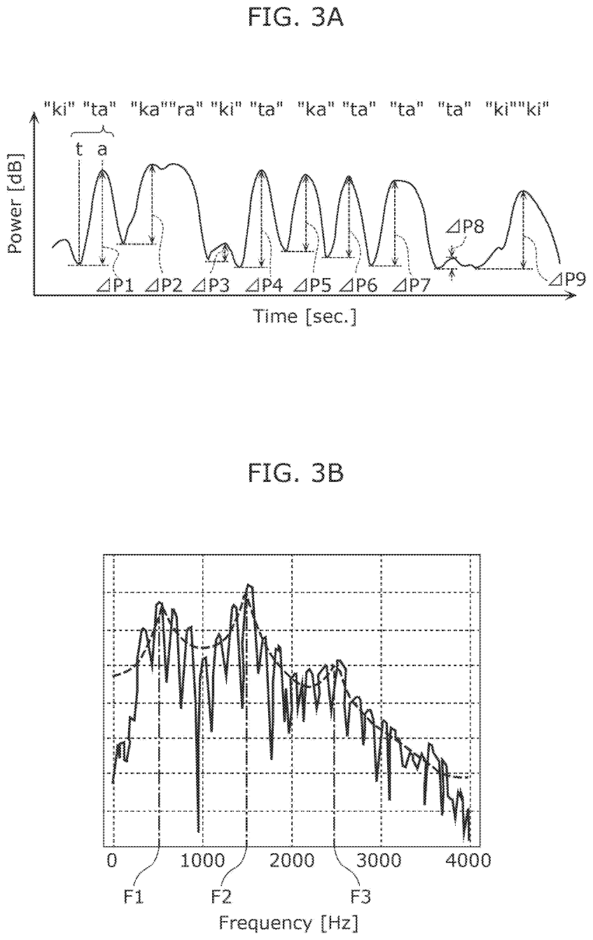

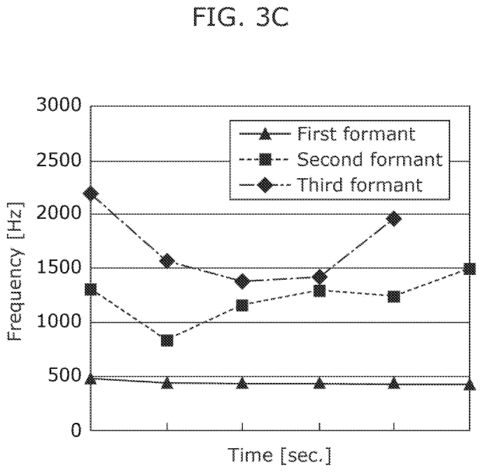

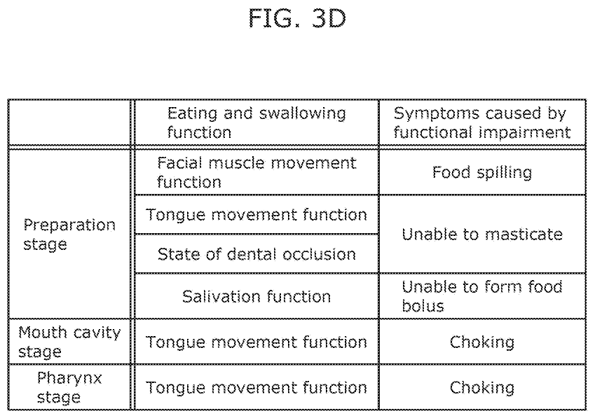

For example, in the case where the speech data recorded by sound recorder 35 is speech data obtained from a speech obtained as a result of the subject speaking a predetermined syllable that is composed of a consonant and a vowel that follows the consonant, outputter 25 calculates a difference between sound pressures of the consonant and the vowel as a feature value. This will be described with reference to FIG. 3A.

FIG. 3A is a diagram showing an example of speech data indicating a speech spoken by the subject. Specifically, FIG. 3A is a graph showing speech data obtained as a result of the subject speaking “ki ta ka ra ki ta ka ta ta ta ki ki”. In the graph shown in FIG. 3A, the horizontal axis indicates time, and the vertical axis indicates power (sound pressure). The power indicated by the vertical axis in FIG. 3A is expressed in the unit of decibel (dB).

In the graph shown in FIG. 3A, variation in sound pressure that corresponds to “ki”, “ta”, “ka”, “ra”, “ki”, “ta”, “ka”, “ta”, “ta”, “ta”, “ki”, and “ki” is observed. Sound recorder 35 records data shown in FIG. 3A as the speech data from the subject. Outputter 25 calculates, by using, for example, a known method, the sound pressure of each of “k” and “i” included in “ki” recorded in the speech data shown in FIG. 3A, the sound pressure of each of “t” and “a” included in “ta” recorded in the speech data, and the sound pressure of each of “k” and “a” included in “ka” recorded in the speech data. Also, when the subject speaks “ki ta ka ze to ta i yo u”, outputter 25 calculates the sound pressure of each of “z” and “e” included in “ze”. Outputter 25 calculates, from the calculated sound pressures of “t” and “a”, ΔP1, ΔP4, ΔP6, ΔP7, and ΔP8 that are the differences between the sound pressures of “t” and “a” as feature values. Likewise, outputter 25 calculates ΔP3 and ΔP9 that are the differences between the sound pressures of “k” and “i”, ΔP2 and ΔP5 that are the differences between the sound pressures of “k” and “a”, and the difference between the sound pressures of “z” and “e” (not shown) as feature values.

For example, outputter 25 references reference data that includes threshold values corresponding to the differences between sound pressures and is stored in server storage 23, and evaluates the eating and swallowing function of the subject by determining whether each of the differences between sound pressures is greater than or equal to the corresponding threshold value.

For example, in order to pronounce “ki” and “ka”, it is necessary to bring the root of the tongue into contact with the soft palate. By evaluating the function of bringing the root of the tongue into contact with the soft palate (a difference between the sound pressures of “k” and “i”, and a difference between the sound pressures of “k” and “a”), the tongue movement function (including tongue pressure, and the like) in the preparation stage, the mouth cavity stage, and the pharynx stage can be evaluated.

For example, in order to pronounce “ta”, it is necessary to bring the tip of the tongue into contact with the upper jaw on the rear of the front teeth. By evaluating the function of bringing the tip of the tongue into contact with the upper jaw on the rear of the front teeth (a difference between the sound pressures of “t” and “a”), the tongue movement function in the preparation stage and the mouth cavity stage can be evaluated.

For example, in order to pronounce “ze”, it is necessary to bring the tip of the tongue into contact with or close proximity to the upper front teeth. The side surfaces of the tongue need to be supported by the row of teeth, which means the presence of teeth is important. By evaluating the presence of the row of teeth including the upper front teeth (a difference between the sound pressures of “z” and “e”), the state of dental occlusion in the preparation stage can be evaluated such as estimating the number of remaining teeth, and determining the influence on the mastication ability if the number of remaining teeth is small.

Also, for example, in the case where the speech data recorded by sound recorder 35 is speech data obtained from a speech obtained as a result of the subject speaking a predetermined sentence that includes a syllable part that includes a consonant, a vowel that follows the consonant, and a consonant that follows the vowel, outputter 25 calculates the time required for the subject to pronounce the syllable part as a feature value.

For example, in the case where the subject speaks a predetermined sentence that includes “kaze”, the predetermined sentence includes a syllable part that includes a consonant “k”, a vowel “a” that follows the consonant, and a consonant “z” that follows the vowel. Outputter 25 calculates the time required for the subject to pronounce the syllable part composed of “k-a-z” as a feature value.

For example, outputter 25 references reference data that includes a threshold value that corresponds to the time required for the subject to pronounce the syllable part, and evaluates the eating and swallowing function of the subject by determining whether the time required for the subject to pronounce the syllable part is greater than or equal to the threshold value.

For example, the time required for the subject to pronounce a syllable part that is composed of a consonant, a vowel, and a consonant varies according to the tongue movement function (tongue dexterity, tongue pressure, or the like) of the subject. By evaluating the time required for the subject to pronounce the syllable part, the tongue movement function in the preparation stage can be evaluated.

Also, for example, in the case where the speech data recorded by sound recorder 35 is speech data obtained from a speech obtained as a result of the subject speaking a predetermined sentence that includes a character string that includes successive syllables that include vowels, outputter 25 calculates the amount of variation in a first formant frequency, a second formant frequency, or the like obtained from the spectrum of vowel portions as a feature value, and calculates variation in the first formant frequency, the second formant frequency, or the like obtained from the spectrum of vowel portions as a feature value.

It is known that the first formant frequency is a peak frequency of amplitude observed first from the low frequency side of the human speech, and the features of tongue movements (in particular, up down movement) are likely to be reflected. In addition, it is also known that the features of opening movement of the jaws are also likely to be reflected.

It is known that the second formant frequency is a peak frequency of amplitude observed second from the low frequency side of the human speech, and among the resonances generated by the vocal cord sound source in the vocal tract, the mouth cavity such as the lips or the tongue, the nasal cavity, and the like, the influence of the position of the tongue (in particular, the forward and backward position) is likely to be reflected. Also, for example, when the mouth has absent teeth, it is not possible to speak properly. Accordingly, it is considered that the state of dental occlusion (the number of teeth) in the preparation stage affects the second formant frequency. Also, for example, when the mouth produces less saliva, it is not possible to speak properly. Accordingly, it is considered that the salivation function in the preparation stage affects the second formant frequency. The tongue movement function, the salivation function, or the state of dental occlusion (the number of teeth) may be calculated from any of the feature values obtained from the first formant frequency and the second formant frequency.

FIG. 3B is a frequency spectrum diagram illustrating formant frequencies. In the graph shown in FIG. 3B, the horizontal axis indicates frequency [Hz], and the vertical axis indicates amplitude.

As indicated by a broken line in FIG. 3B, a plurality of peaks are observed in data obtained by converting the horizontal axis of the speech data to frequency. Among the plurality of peaks, the frequency of the lowest frequency peak is first formant frequency F1. The frequency of the second lowest frequency peak next to first formant frequency F1 is second formant frequency F2. The frequency of the third lowest frequency peak nest to second formant frequency F2 is third formant frequency F3. In this way, outputter 25 calculates the spectrum of vowel portions by extracting the vowel portions from the speech data recorded by sound recorder 35 using a known method and converting the speech data of the extracted vowel portions to amplitude relative to frequency, and calculates formant frequencies obtained from the spectrum of vowel portions.

The graph shown in FIG. 3B is calculated by converting the speech data recorded from the subject to amplitude data relative to frequency, and obtaining an envelope curve thereof. For calculation of the envelope curve, for example, cepstrum analysis, linear predictive coding (LPC), or the like is used.

FIG. 3C is a diagram showing an example of variation in formant frequencies with time. Specifically, FIG. 3C is a graph illustrating an example of variation in first formant frequency F1, second formant frequency F2, and third formant frequency F3 with time.

For example, the subject is caused to speak a syllable that includes a plurality of successive vowels such as “a i u e o”. Outputter 25 calculates, from speech data indicating the speech spoken by the subject, first formant frequency F1 and second formant frequency F2 of the plurality of vowels. Furthermore, outputter 25 calculates, as feature values, the amount of variation (the amount of variation with time) in first formant frequency F1 and the amount of variation (the amount of variation with time) in second formant frequency F2, first formant frequency F1 and second formant frequency F2 each being the formant frequency of a character string that includes successive vowels.

For example, outputter 25 references reference data that includes a threshold value that corresponds to the amount of variation, and evaluates the eating and swallowing function of the subject by determining whether the amount of variation is greater than or equal to the threshold value.

When looking at first formant frequency F1, it shows, for example, the opening movement of the jaws, or in other words, it shows that the movement of the jaws is impaired in the preparation stage, the mouth cavity stage, and the pharynx stage that are affected by the movement of the jaws. When looking at second formant frequency F2, it shows that second formant frequency F2 is affected by the forward and backward position of the tongue, and the tongue movement in the preparation stage, the mouth cavity stage, and the pharynx stage that are affected by the tongue movement is impaired. When looking at second formant frequency F2, it shows, for example, that the mouth has absent teeth, and it is therefore not possible to speak properly, or in other words, it shows that the state of dental occlusion in the preparation stage is deteriorated. Also, when looking at second formant frequency F2, it shows, for example, that the mouth produces less saliva, and it is therefore not possible to speak properly, or in other words, it shows that the salivation function in the preparation stage is impaired. That is, by evaluating the amount of variation in second formant frequency F2, the salivation function in the preparation stage can be evaluated.

Also, outputter 25 calculates the variation in first formant frequency F1 of a character string that includes successive vowels, as a feature value. For example, in the case where the speech data includes n vowels (where n is a natural number), n first formant frequencies F1 are obtained, and the variation in first formant frequency F1 is calculated by using all or some of n first formant frequencies F1. The degree of variation calculated as a feature value is, for example, a standard deviation.

For example, outputter 25 references reference data that includes a threshold value that corresponds to the variation, and evaluates the eating and swallowing function of the subject by determining whether the variation is greater than or equal to the threshold value.

The fact that the variation in first formant frequency F1 is large (is greater than or equal to the threshold value) indicates that, for example, the up down movement of the tongue is slow, or in other words, it shows that the tongue movement function of pushing the tip of the tongue against the upper jaw to transfer the food bolus into the pharynx in the mouth cavity stage is impaired. That is, by evaluating the variation in first formant frequency F1, the tongue movement function in the mouth cavity stage can be evaluated.

Also, for example, outputter 25 calculates the pitch (tone) of a speech of the subject obtained as a result of the subject speaking a predetermined syllable or a predetermined sentence as a feature value.

For example, outputter 25 references reference data that includes a threshold value that corresponds to the pitch, and evaluates the eating and swallowing function of the subject by determining whether the pitch is greater than or equal to the threshold value.

Also, for example, in the case where the speech data recorded by sound recorder 35 is speech data obtained from a speech obtained as a result of the subject speaking a predetermined sentence that includes a predetermined word, outputter 25 calculates the time required for the subject to pronounce the predetermined word as a feature value.

For example, in the case where the subject speaks a predetermined sentence that includes “ta i yo u”, the subject first recognizes that “taiyou (that means the sun in Japanese)” included in a character string of “ta i yo u” is a word, and then speaks the character string of “ta i yo u”. If it takes time to pronounce the predetermined word, the subject may be suffering from dementia. Here, it is said that the number of teeth affects dementia. This is because the number of teeth affects the activity of the brain, and when the number of teeth decreases, stimuli to the brain decrease, which increases the risk of developing dementia. That is, the fact that the subject may be suffering from dementia corresponds to the number of teeth, and also corresponds to the state of dental occlusion for crushing and grinding food in the preparation stage. Accordingly, the fact that the time required for the subject to pronounce a predetermined word is large (is greater than or equal to the threshold value) means that the subject may be suffering from dementia. In other words, it indicates that the state of dental occlusion in the preparation stage is deteriorated. That is, by evaluating the time required for the subject to pronounce a predetermined word, the state of dental occlusion in the preparation stage can be evaluated.

Outputter 25 may calculate the time required for the subject to pronounce an entire predetermined sentence as a feature value. In this case as well, by evaluating the time required for the subject to pronounce an entire predetermined sentence in the same manner, the state of dental occlusion in the preparation stage can be evaluated. Also the tongue movement (dexterity) can also be evaluated in the same manner. That is, by evaluating the time required for the subject to pronounce an entire predetermined sentence, the tongue movement in the preparation stage can be evaluated.

Also, for example, in the case where the speech data recorded by sound recorder 35 is speech data obtained from a speech obtained as a result of the subject speaking a predetermined sentence that includes a phrase in which a syllable composed of a stop consonant and a vowel that follows the stop consonant is repeated, outputter 25 calculates the number of times the syllable was repeatedly spoken within a predetermined length of time (for example, five seconds, or the like) as a feature value.

For example, outputter 25 references reference data that includes a threshold value that corresponds to the number of times the syllable was repeatedly spoken, and evaluates the eating and swallowing function of the subject by determining whether the number of times the syllable was repeatedly spoken is greater than or equal to the threshold value.

For example, it is assumed that the subject speaks a predetermined sentence that includes a phrase in which a syllable composed of a consonant and a vowel that follows the consonant is repeated such as “pa pa pa pa pa . . . ”, “ta ta ta ta ta . . . ”, “ka ka ka ka ka . . . ”, or “ra ra ra ra ra . . . ”.

For example, in order to pronounce “pa”, it is necessary to open and close the mouth (lips) in the up down direction. When the function of opening and closing the lips in the up down direction is impaired, it is not possible to quickly pronounce “pa” a predetermined number of times (threshold value) or more during a predetermined length of time. The movement of opening and closing the lips in the up down direction is similar to the movement of moving the food into the mouth cavity without spilling in the preparation stage. For this reason, the function of quickly pronouncing “pa”, or in other words, the function of quickly and repeatedly opening and closing the lips in the up down direction corresponds to the facial muscle movement function of moving the food into the mouth cavity without spilling the food in the preparation stage. That is, by evaluating the number of times “pa” was spoken within a predetermined length of time, the facial muscle movement function in the preparation stage can be evaluated.

For example, in order to pronounce “ta”, as described above, it is necessary to bring the tip of the tongue into contact with the upper jaw on the rear of the front teeth. The movement of bringing the tip of the tongue into contact with the upper jaw on the rear of the front teeth is similar to the movement of pushing the food against the teeth and mixing the finely crushed food with saliva to gather the food into a food bolus, performed in the preparation stage, and the movement of raising the tongue (the tip of the tongue) to move the food bolus from the mouth cavity to the pharynx, performed in the mouth cavity stage. For this reason, the function of quickly pronouncing “ta”, or in other words, the function of quickly and repeatedly bringing the tip of the tongue into contact with the upper jaw on the rear of the front teeth corresponds to the tongue movement function of pushing the food against the teeth and mixing the finely crushed food with saliva to gather the food into a food bolus, performed in the preparation stage, and the tongue movement function of moving the food bolus to the pharynx, performed in the mouth cavity stage. That is, by evaluating the number of times “ta” was spoken within a predetermined length of time, the tongue movement function in the preparation stage and the tongue movement function in the mouth cavity stage can be evaluated.

For example, in order to pronounce “ka”, in the same manner as when “ki” is pronounced described above, it is necessary to bring the root of the tongue into contact with the soft palate. The movement of bringing the root of the tongue into contact with the soft palate is similar to the movement of passing the food bolus through the pharynx (swallowing), performed in the mouth cavity stage and the pharynx stage. Furthermore, when ingesting food or drink into the mouth (preparation stage), and when masticating the food in the mouth to form a food bolus (preparation stage), the root of the tongue comes into contact with the soft palate to perform the movement of preventing the food bolus from fluxing into the pharynx and the movement of preventing choking. This is similar to the tongue movement performed when pronouncing “k”. That is, by evaluating the number of times “ka” was spoken within a predetermined length of time, the tongue movement function in the preparation stage, the mouth cavity stage, and the pharynx stage can be evaluated. Also, the tongue movement function corresponds to the function of preventing the food from fluxing into the pharynx and the function of preventing choking.

For example, in order to pronounce “ra”, it is necessary to curve the tongue. The movement of curving the tongue is similar to the movement of mixing the food with saliva to form a food bolus, performed in the preparation stage. For this reason, quickly pronouncing “ra”, or in other words, the function of curving the tongue quickly and repeatedly corresponds to the tongue movement function of mixing the food with saliva to form a food bolus, performed in the preparation stage. That is, by evaluating the number of times “ra” was spoken within a predetermined length of time, the tongue movement function in the preparation stage can be evaluated.

As described above, outputter 25 evaluates the eating and swallowing function of the subject while distinguishing between the preparation stage, the mouth cavity stage, and the pharynx stage, such as, for example, the tongue movement function in the preparation stage and the tongue movement function in the mouth cavity stage. For example, outputter 25 references reference data that includes a correspondence relationship between the type of feature value and the eating and swallowing function in at least one of the preparation stage, the mouth cavity stage, and the pharynx stage. For example, when attention is given to the time required for the subject to pronounce a syllable part that is composed of “k-a-z” as a feature value, the time required for the subject to pronounce a syllable part that is composed of “k-a-z” is associated with the tongue movement function in the preparation stage. Accordingly, outputter 25 can evaluate the eating and swallowing function of the subject while distinguishing between the preparation stage, the mouth cavity stage, and the pharynx stage. By evaluating the eating and swallowing function of the subject while distinguishing between the preparation stage, the mouth cavity stage, and the pharynx stage, it is possible to predict which symptoms will occur in the subject. This will be described with reference to FIG. 3D.

FIG. 3D is a diagram showing specific examples of eating and swallowing functions in the preparation stage, the mouth cavity stage, and the pharynx stage and symptoms that occur when the functions are impaired.

When the facial muscle movement function in the preparation stage is impaired, in eating and swallowing, a symptom of spilling food while eating the food is observed. When the tongue movement function and the state of dental occlusion in the preparation stage are deteriorated, a symptom of not being able to properly masticate the food (crush and grind the food) is observed in eating and swallowing. When the salivation function in the preparation stage is impaired, a symptom of not being able to form a food bolus and thus the food remains in separate pieces is observed in eating and swallowing. Also, when the tongue movement function in the mouth cavity stage and the pharynx stage is impaired, a symptom of not being able to properly swallow the food bolus through the pharynx to the esophagus, causing choking is observed in eating and swallowing.

As described above, because which symptoms will occur when the eating and swallowing functions in each stage are impaired are known, by evaluating the eating and swallowing function of the subject while distinguishing between the preparation stage, the mouth cavity stage, and the pharynx stage, it is possible to output a recipe adjusted to be suitable for the corresponding eating and swallowing function of the subject, which will be described below.

Outputter 25 outputs ability information as a result of evaluation of the eating and swallowing function of the evaluated subject. Also, outputter 25 outputs a recipe for cooking the dish based on the ability information of the evaluated subject. Outputter 25 may output the ability information to information terminal 30. In this case, outputter 25 outputs the ability information to terminal communicator 33 by using, for example, wired communication or wireless communication via server communicator 22. The ability information output to information terminal 30 in this way is displayed on a display or the like for the subject and the like.

Accordingly, it is desirable that the eating and swallowing function shown in the ability information is presented in an easy-to-understand manner for the subject and the like. In the present embodiment, the eating and swallowing function is classified into the following six items: the biting ability, the tongue movement, the swallowing action, the ability to gather food into a food bolus, the movement of the jaws, and the ability to prevent choking of the subject.

Specifically, “the ability to eat solid food (or in other words, the ability to bite solid food)” is comprehensively quantified mainly based on the state of dental occlusion in the preparation stage, and the mastication muscle movement function, the facial muscle movement function, and the tongue dexterity in the preparation stage. Also, “the tongue movement” is comprehensively quantified mainly based on the tongue movement function in the pharynx stage, the tongue movement function in the preparation stage, and the tongue movement function in the mouth cavity stage. Also, “the swallowing action” is comprehensively quantified mainly based on the soft palate raising function in the mouth cavity stage and the tongue movement function in the mouth cavity stage and the pharynx stage. Also, “the ability to gather food into a food bolus” is comprehensively quantified mainly based on the tongue movement function in the preparation stage, the cheek movement function in the preparation stage, and the salivation function in the preparation stage. Also, “the movement of the jaws” is comprehensively quantified mainly based on the jaws movement function in the preparation stage, the facial muscle movement function, and the mastication muscle movement function in the preparation stage. Also, “the ability to prevent choking” is comprehensively quantified mainly based on the tongue movement function in the preparation stage, the mouth cavity stage, and the pharynx stage.

Also, outputter 25 may be configured to comprehensively evaluate the eating and swallowing function for the above-described comprehensively quantified six items by referencing reference data that includes threshold values for the six items.

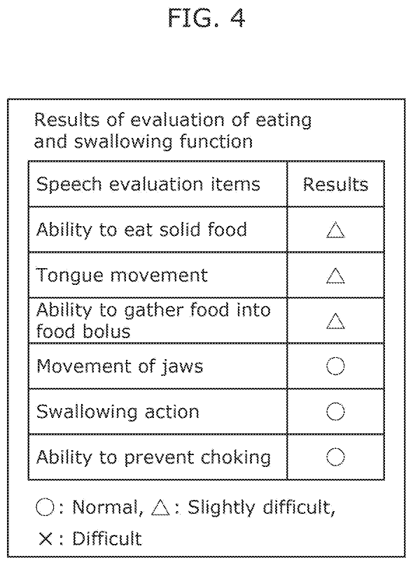

FIG. 4 is a diagram showing an example of the ability information. The image data of an image that corresponds to the ability information displayed on information terminal 30 may be, for example, a table as shown in FIG. 4. FIG. 4 shows the results of evaluation of the eating and swallowing function of the subject in terms of the following six items: “the ability to eat solid food”, “the tongue movement”, “the ability to gather food into a food bolus”, “the movement of the jaws”, “the swallowing action”, and “the ability to prevent choking”.

For example, the ability information is the result of evaluation indicated in three levels by using a circle mark, a triangle mark, and a cross mark. The circle mark indicates “normal”, the triangle mark indicates “slightly difficult”, and the cross mark indicates “difficult”. The results of evaluation shown in FIG. 4 indicate that the subject has slight difficulties in terms of the following three items: “the ability to eat solid food”; “the tongue movement”; and “the ability to gather food into a food bolus”, from which it can be seen that the subject is at the second level out of the three levels and has slight difficulties.

The result of evaluation does not necessarily need to be indicated in three levels, and may be indicated in two levels, or four or more levels. That is, the number of threshold values corresponding to each item included in the reference data is not limited two, and may be one, or three or more. Specifically, if a feature value is greater than or equal to a first threshold value, the result of evaluation is determined as normal. If the feature value is less than the first threshold value and greater than a second threshold value, the result of evaluation is determined as slightly difficult. If the feature value is less than the second threshold value and greater than a third threshold value, the result of evaluation is determined as difficult. If the feature value is less than or equal to the third threshold value, the result of evaluation is determined as very difficult. The results of evaluation may be indicated by using OK (normal) and NG (abnormal) instead of using a circle mark and a cross mark. Also, when displaying the ability information on a display, only items of the eating and swallowing function that may be impaired can be displayed. That is, in the example shown in FIG. 4, only “the ability to eat solid food”, “the tongue movement”, and “the ability to gather food into a food bolus” may be displayed.

### [Processing Procedure for Outputting Recipe]

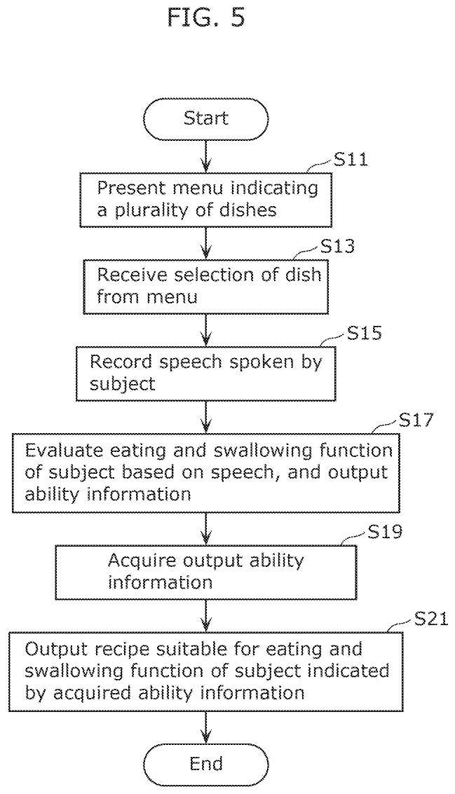

Next, a processing procedure for outputting a recipe performed by recipe output system 100 will be described. FIG. 5 is a flowchart illustrating a processing procedure for outputting a recipe according to the embodiment.

As shown in FIG. 5, recipe output system 100 first presents a menu indicating a plurality of dishes to a subject or a food preparer who cooks food for the subject (step S11). The menu may be presented by, for example, accessing server device 20 by using information terminal 30, and displaying the menu stored in server storage 23 on a display.

The subject or the food preparer selects one from among the plurality of dishes included in the presented menu based on the criteria such as what the subject wants to eat, or what is good for the subject to eat. The selection of a dish is received by recipe output system 100 as a result of, for example, the name of the dish displayed on a touch panel display being tapped (step S13). The selection of the dish received is transmitted to server device 20 as dish information indicating the selected dish. The selections of a plurality of dishes may be received by successively receiving the selection of a dish. That is, the selections of a plurality of dishes selected for one meal to be eaten by the subject may be collectively received. The collective selection of a plurality of dishes received may be used as information added to an output recipe.

Here, as described above, a speech used to evaluate the eating and swallowing function of the subject is recorded (step S15). As described above, the speech is recorded as a result of sound recorder 35 of information terminal 30 recording a speech spoken by the subject as speech data. The speech data recorded by information terminal 30 is transmitted to server device 20.

Next, recipe output system 100 evaluates the eating and swallowing function of the subject based on the speech. Specifically, in recipe output system 100, as described above, the eating and swallowing function of the subject is evaluated based on the speech data transmitted to server device 20, and ability information that is the result of evaluation is output (step S17).

The ability information output in this way is acquired by acquirer 24 (step S19). In the case where, for example, ability information, which is the results of evaluation of the eating and swallowing function of the subject based on the speeches spoken by the subject in the past, is stored in server storage 23 or the like, the ability information may be used. Specifically, acquirer 24 acquires the ability information stored in server storage 23. Accordingly, in this case, steps S15 and S17 may be omitted.

Outputter 25 outputs a recipe that is suitable for the eating and swallowing function of the subject based on the eating and swallowing function of the subject indicated by the acquired ability information (step S21). The output recipe is transmitted to information terminal 30 as recipe information indicating the recipe.

Hereinafter, the processing procedure performed in step S21 will be described in detail.

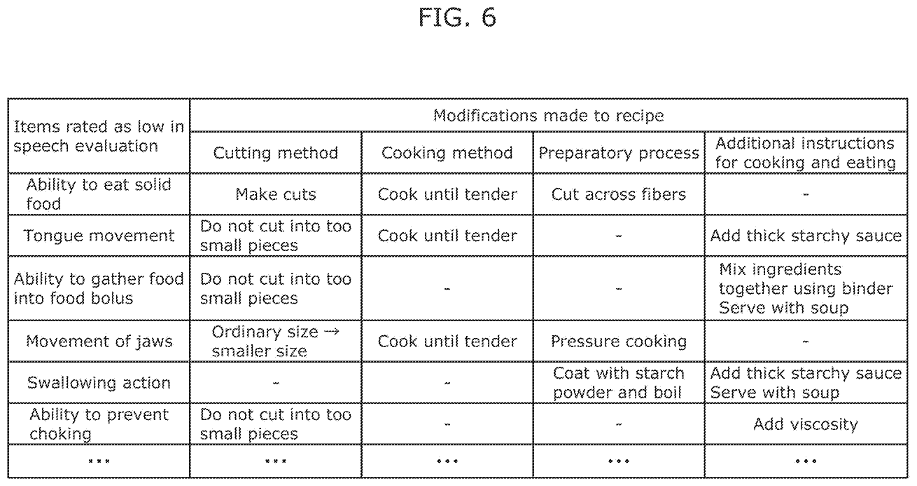

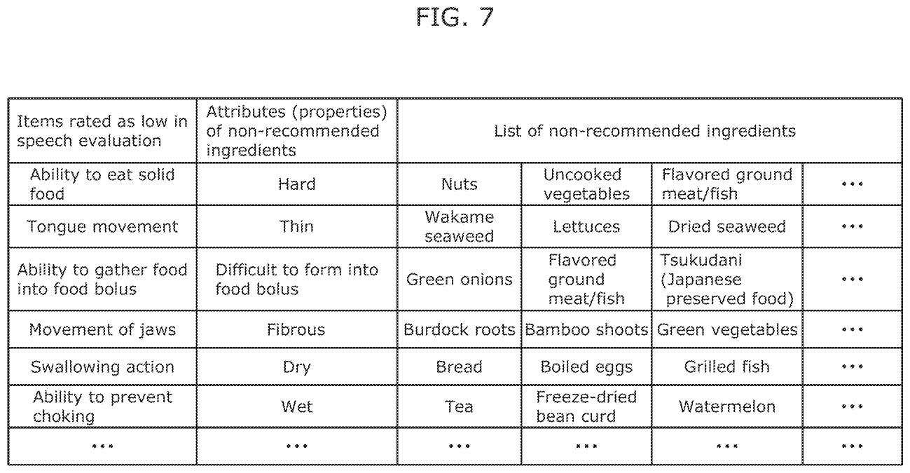

In the outputting a recipe that is suitable for the eating and swallowing function of the subject performed in step S21, for the items of the eating and swallowing function for which abnormal symptoms are observed, reference is made to modification tables as shown in FIGS. 6 and 7 in which eating and swallowing functions and modifications to be made to an output recipe are associated with each other, then, modifications are made to a standard recipe, and a recipe that has been adjusted to be suitable for the eating and swallowing function of the subject is output.

FIG. 6 is a first modification table in which eating and swallowing functions and modifications to be made to an output recipe are associated with each other. FIG. 6 shows “the cutting method (of ingredients)”, “the cooking method”, “the method of preparatory process”, and “the additional instructions for cooking and eating” that will be modified in an output recipe in the case where abnormal symptoms are observed in the following functions: “the ability to eat solid food”, “the tongue movement”, “the ability to gather food into a food bolus”, “the movement of the jaws”, “the swallowing action, and “the ability to prevent choking”.

For example, in the case where “the ability to eat solid food” is determined as not normal, an instruction such as “make cuts” is added to “the cutting method” in the recipe. Also, an instruction such as “cook until tender” is added to “the cooking method” in the recipe. Also, an instruction such as “cut across fibers” is added to “the preparatory process” in the recipe. For example, in the case where “the tongue movement” is determined as not normal, an instruction such as “do not cut into too small pieces” is added to “the cutting method” in the recipe. Also, an instruction such as “cook until tender” is added to “the cooking methods” in the recipe. Also, an instruction such as “add thick starchy sauce” is added to “the additional instructions for cooking and eating” in the recipe.

In this way, in the modification table as shown in FIG. 6, in the case where the eating and swallowing function if the subject is determined as not normal, only modification points that need to be modified are associated with the eating and swallowing functions. The standard recipe before modifications are made is a recipe for cooking food for a healthy person with normal eating and swallowing abilities. As the standard recipe, one of a plurality of recipes stored in, for example, server storage 23 as a recipe database that corresponds to the selected dish may be used, or a recipe that corresponds to the selected dish acquired from an external recipe providing service or the like may be used. In this case, the instructions in the following items that are included in the acquired standard recipe: “the cutting method”, “the cooking method”, “the preparatory process”, and “the additional instructions for cooking and eating” are written differently from recipe to recipe, and thus language recognition that uses machine learning or the like may be used to match between modification points that need to be modified and modified instructions.

Also, for example, in the case where both “the ability to eat solid food” and “the tongue movement” are determined as not normal, for example, “cook until tender” is written in “the cooking method” for the both items, but it does not mean that the heating time needs to be extended as compared with the case where either one of “the ability to eat solid food” or “the tongue movement” is determined as not normal. That is, “the cooking method” is modified to “cook until tender” when any one of “the ability to eat solid food”, “the tongue movement”, and “the movement of the jaws”, for which “cook until tender” is written in “the cooking method” as a modification point, is determined as not normal.

In the manner as described above, outputter 25 outputs a recipe that has been adjusted to be suitable for the eating and swallowing function of the subject.

Also, FIG. 7 is a second modification table in which eating and swallowing functions and modifications to be made to an output recipe are associated with each other. FIG. 7 shows a list indicating ingredients to be modified in an output recipe in the case where abnormal symptoms are observed in the following functions: “the ability to eat solid food”, “the tongue movement”, “the ability to gather food into a food bolus”, “the movement of the jaws”, “the swallowing action”, and “the ability to prevent choking”.

Specifically, FIG. 7 shows a list indicating non-recommended ingredients that are not recommended to be used to cook food according to the eating and swallowing abilities determined as not normal. For example, it can be seen from the list that, in the case of a subject whose “ability to eat solid food” is determined as not normal, the use of “nuts”, “uncooked vegetables”, “flavored ground meat/fish”, and the like is not recommended. Likewise, it can be seen from the list that, in the case of a subject whose “tongue movement” is determined as not normal, the use of “wakame seaweed”, “lettuces”, “dried seaweed”, and the like is not recommended. In the case where the standard recipe includes ingredients that are included in the list indicating non-recommended ingredients, a recipe obtained by removing the ingredients that are included in the list indicating non-recommended ingredients from the standard recipe is output. Also, in the case where it is possible to present alternative ingredients to the non-recommended ingredients, a recipe obtained by replacing the non-recommended ingredients in the standard recipe with alternative ingredients is output.

FIG. 7 also shows the attributes (properties) of non-recommended ingredients to cover entire non-recommended ingredients for each of the eating and swallowing abilities. With the attributes of non-recommended ingredients, the food preparer or the like can determine by himself/herself whether or not to use an ingredient that is not included in the list but may not be recommended. That is, by adding the attributes of non-recommended ingredients as described above to the recipe as notes, the use of ingredients that are not recommended to use when cooking food can be avoided based on the decision of the food preparer.

In the manner as described above, outputter 25 outputs a recipe for cooking the selected dish by using ingredients that are suitable for the eating and swallowing function of the subject.

In the case where outputter 25 cannot output a recipe for cooking the dish suitable for the eating and swallowing function of the subject, outputter 25 may output a list indicating a plurality of recommended dishes instead of the recipe for cooking the dish. The list indicating a plurality of recommended dishes is displayed on information terminal 30. Outputter 25 outputs a recipe for cooking a recommended dish for which a selection from the list indicating a plurality of recommended dishes was received from the subject, the food preparer, or the like. At this time, outputter 25 outputs a recipe for cooking the recommended dish that has been adjusted to be suitable for the eating and swallowing function of the subject in the same manner as described above.

### EXAMPLES

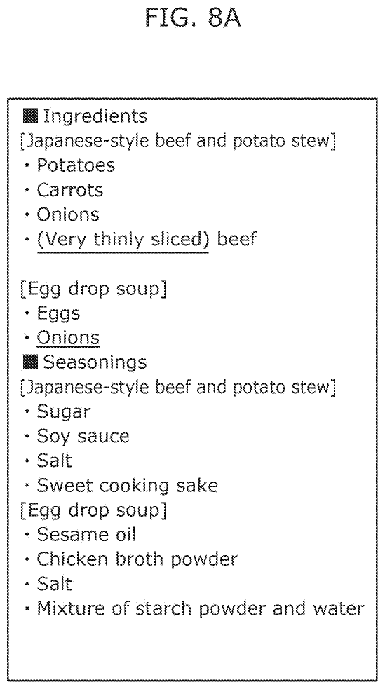

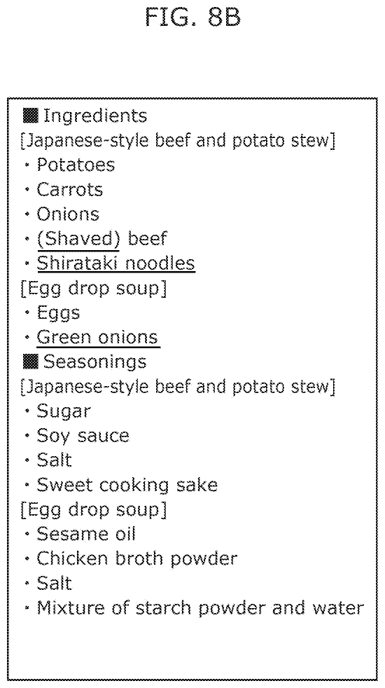

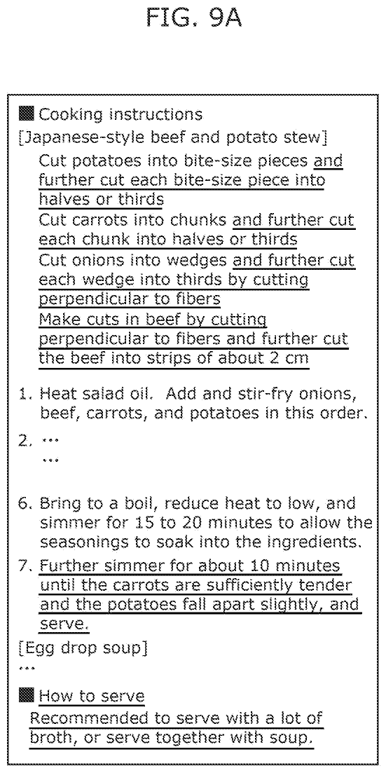

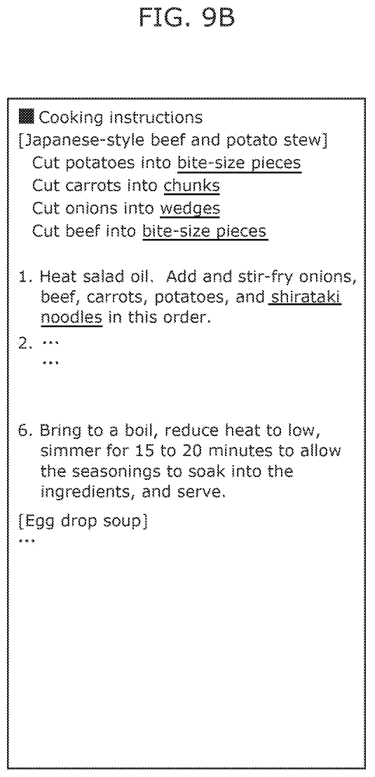

Hereinafter, examples of the embodiment will be described with reference to FIGS. 8A to 9B. FIG. 8A is a first diagram showing an example of an output recipe according to an example of the embodiment. FIG. 8B is a first diagram showing an example of an output recipe according to a comparative example. FIG. 9A is a second diagram showing an example of an output recipe according to the example of the embodiment. FIG. 9B is a second diagram showing an example of an output recipe according to the comparative example.

FIGS. 8A to 9B show output recipes according to the example and the comparative example. The output recipe according to the example is a recipe that is output assuming that the subject is a person who exhibited the ability information shown in FIG. 4. Also, the output recipe according to the comparative example is a recipe that is output assuming that the subject is a healthy person with no abnormal symptoms being observed in the eating and swallowing function. That is, FIGS. 8B and 9B show a standard recipe. In this example, “Japanese-style beef and potato stew” and “egg drop soup” have been selected, and a recipe for cooking these is output. Also, in the diagrams, modifications made in the example and the comparative example are underlined.

In FIG. 4, the eating and swallowing function of the subject is evaluated based on three levels. When the eating and swallowing function is represented by a triangle mark indicating “slightly difficult” or a cross mark indicating “difficult”, the recipe is modified. For example, the recipe may be modified according to the number of levels into which the eating and swallowing function of the subject is evaluated. That is, in the case where the eating and swallowing function of the subject is evaluated based on three levels: “normal”, “slightly difficult”, and “difficult”, for example, “the cutting method” may be modified based on three levels that correspond to the three levels of the eating and swallowing function, such as “no additional process”, “make cuts”, and “cut into smaller pieces”.

In the food items shown as the ingredients, “(shaved) beef” written in the standard recipe has been modified to “(very thinly sliced) beef” in the recipe output in the example. By using very thinly sliced beef, which is thinner than shaved beef, as beef, a modification suitable for “the ability to eat solid food” of the subject is made.

Also, in the food items shown as the ingredients, “shirataki noodles” written in the standard recipe is deleted in the recipe output in the example. Shirataki noodles is a food item that has the property of not being softened even when it is heated and being difficult to form into a food bolus after it is crushed and ground by the teeth, and it is therefore necessary to finely crush and grind by the teeth, and is also a food item that is elastic and is similar to konnyaku that needs to be appropriately guided to the mastication position by the tongue. That is, this modification has been made so as to be suitable for “the tongue movement” of the subject.

Also, in the food items shown as the ingredients, “green onions” written in the standard recipe has been modified to “onions” in the recipe output in the example. Green onions is a food item that is fibrous and solid and is difficult to finely grind by the teeth. That is, this modification has been made so as to be suitable for “the ability to bite solid food” of the subject.

Also, in the cooking instructions, “cut potatoes into bite-size pieces” written in the standard recipe has been modified to “cut potatoes into bite-size pieces and further cut each bite-size piece into halves or thirds” in the recipe output in the example. By cutting the food item from a standard size to a smaller size, the burden during mastication is reduced. That is, this modification has been made so as to be suitable for “the ability to eat solid food” of the subject. The same applies to the modification made on the cutting method of carrots.

Also, in the cooking instructions, “cut onions into wedges” written in the standard recipe has been modified to “cut onions into wedges and further cut each wedge into thirds by cutting perpendicular to fibers” in the recipe output in the example. By cutting the food item from a standard size to a smaller size and cutting across the fibers, the burden during mastication is further reduced. That is, this modification has been made so as to be suitable for “the ability to eat solid food” of the subject. The same applies to the modification made on the cutting method of beef.

Also, in the cooking instructions, an additional cooking instruction “further simmer for about 10 minutes until the carrots are sufficiently tender and the potatoes fall apart slightly, and serve”, which was not written in the standard recipe, has been added after step 6 in the recipe output in the example. This instruction has been added to cook relatively hard root vegetables until they are tender. That is, this modification has been made so as to be suitable for “the ability to eat solid food” of the subject.

Also, an additional instruction “recommended to serve with a lot of broth, or serve together with soup”, which was not written in the standard recipe, has been added to “how to serve” in the recipe output in the example. This instruction has been added to externally provide additional moisture to aid salivation when eating low-moisture content food. Also, in this example, the soup (specifically, egg drop soup) selected by the subject includes starch powder, and thus an advantageous effect of increasing fluid viscosity during mastication can also be expected. That is, recipe output system 100 may be configured to add additional instructions made by taking into consideration the ingredients used to cook the dishes collectively selected by the subject. This modification has been made so as to be suitable for “the tongue movement” and “the ability to gather food into a food bolus” of the subject.

In the manner as described above, the standard recipe is modified to be suitable for the eating and swallowing function of the subject, and a recipe adjusted to be suitable for the eating and swallowing function of the subject is output.

[Advantageous Effects, etc.]

As described above, a recipe output method according to the present embodiment includes: receiving a selection of a dish from a menu indicating a plurality of dishes; acquiring ability information indicating an eating and swallowing function of a subject determined by evaluation based on a speech spoken by the subject; and outputting a recipe for cooking the dish for which the selection was received, the recipe being adjusted to be suitable for the eating and swallowing function of the subject indicated by the ability information acquired.

With the recipe output method described above, the ability information, which is the result of evaluation of the eating and swallowing function of the subject predicted from the speech spoken by the subject, is acquired, and a recipe for cooking the selected dish is output according to the acquired ability information. Accordingly, the recipe is output according to the eating and swallowing function of the subject. Thus, with the recipe output method, the recipe adjusted to be suitable for the subject can be output.

Also, for example, the recipe output method may further include: recording the speech spoken by the subject; and evaluating the eating and swallowing function of the subject based on the speech spoken by the subject recorded and outputting the ability information, and the acquiring the ability information may include acquiring the ability information output.

With this configuration, the speech spoken by the subject is recorded, and the eating and swallowing function of the subject can be evaluated based on the recorded speech. Also, the recipe for cooking the selected dish can be output based on the ability information that is the result of the evaluation of the eating and swallowing function. Accordingly, the recipe is output according to the eating and swallowing function based on the prediction from the recorded speech of the subject. Thus, with the recipe output method, the recipe adjusted to be suitable for the subject can be output.

Also, for example, the eating and swallowing function of the subject may include at least one of the following abilities of the subject: biting ability, tongue movement, swallowing action, ability to gather food into a food bolus, movement of jaws, and ability to prevent choking.

With this configuration, the recipe for cooking the selected dish can be output based on the ability information that is the result of the evaluation of the eating and swallowing function that includes at least one of the following abilities of the subject: the biting ability, the tongue movement, the swallowing action, the ability to gather food into a food bolus, the movement of the jaws, and the ability to prevent choking. Accordingly, the recipe is output according to the eating and swallowing function that includes at least one of the following abilities of the subject: the biting ability of the subject, the tongue movement, the swallowing action, the ability to gather food into a food bolus, the movement of the jaws, and the ability to prevent choking. Thus, with the recipe output method, the recipe adjusted to be suitable for the subject can be output.

Also, for example, the outputting the recipe may further include, when the eating and swallowing function of the subject is less than or equal to a predetermined threshold value, outputting the recipe that includes at least one of the following items suitable for the eating and swallowing function of the subject: a cooking method, an amount of water added, a cutting method for cutting ingredients, a method of preparatory process, and an eating manner.

With this configuration, the recipe in which at least one of the cooking method, the amount of water added, the cutting method for cutting ingredients, the method for preparatory process, and the eating manner is suitable for the eating and swallowing function of the subject can be output. Accordingly, the recipe that includes a specific cooking procedure can be output. Accordingly, with the recipe output method, the dish suitable for the eating and swallowing function of the subject can be easily cooked.

Also, for example, the outputting the recipe may further include, when the eating and swallowing function of the subject is less than or equal to a predetermined threshold value, outputting the recipe for cooking the dish for which the selection was received by using ingredients suitable for the eating and swallowing function of the subject.

With this configuration, the eating and swallowing function of the subject can be easily evaluated by performing numerical comparison relative to a threshold value. Accordingly, with the recipe output method, the processing load during evaluation of the eating and swallowing function can be reduced, and a simple recipe output system can be implemented.

Also, for example, the outputting the recipe may further include outputting the recipe adjusted to be suitable for the eating and swallowing function of the subject by making a modification to a standard recipe for cooking the dish for which the selection was received in a normal way by referencing a modification table in which eating and swallowing functions and modifications to be made in the outputting the recipe are associated with each other.

With this configuration, by simply making modifications to the standard recipe based on the result of the evaluation of the eating and swallowing function of the subject, the recipe adjusted to be suitable for the eating and swallowing function of the subject can be output. Accordingly, with the recipe output method, the processing load during output of the recipe can be reduced, and a simple recipe output system can be implemented.

Also, for example, the outputting the recipe may further include: when the eating and swallowing function of the subject is less than or equal to a predetermined threshold value, presenting a list indicating a plurality of recommended dishes that are recommended instead of the dish for which the selection was received; and outputting a recipe for cooking a recommended dish for which a selection from the list indicating the plurality of recommended dishes was received, the recipe being adjusted to be suitable for the eating and swallowing function of the subject indicated by the ability information acquired.

With this configuration, it is possible to present recommended dishes that can replace the selected dish according to the eating and swallowing function of the subject. By again receiving a selection made from among the recommended dishes presented, a recipe for cooking a dish according to the preference of the subject or the like can be output, as compared with the case where an alternative dish is simply suggested. The recipe output at this time may be a standard cooking recipe suitable for the eating and swallowing function of the subject, or may be a recipe obtained by making modifications to the standard recipe so as to be suitable for the eating and swallowing disorders of the subject. Accordingly, with the recipe output method, a wide variety of recommended dishes can be provided, and a recipe for cooking a dish according to the preference of the subject or the like can be output.

Also, recipe output system 100 according to the present embodiment includes: a receiver (input receiver 32) that receives a selection of a dish from a menu indicating a plurality of dishes; acquirer 24 that acquires ability information indicating an eating and swallowing function of a subject determined by evaluation based on a speech spoken by the subject; and outputter 25 that outputs a recipe for cooking the dish for which the selection was received, the recipe being adjusted to be suitable for the eating and swallowing function of the subject indicated by the ability information acquired.

Recipe output system 100 configured as described above can acquire the ability information that is the result of evaluation of the eating and swallowing function of the subject predicted from the speech spoken by the subject, and output a recipe for cooking the selected dish according to the ability information. Accordingly, the recipe is output according to the eating and swallowing function of the subject. Thus, recipe output system 100 can output the recipe adjusted to be suitable for the subject.

### OTHER EMBODIMENTS

Up to there, the recipe output method and the like according to the embodiment have been described. However, the present invention is not limited to the embodiment given above.

For example, the reference data is data prepared in advance, but may be updated based on the result of evaluation obtained from an actual diagnosis of the eating and swallowing function of the subject performed by an expert. With this configuration, the accuracy of evaluation of the eating and swallowing function can be enhanced, and a recipe that is more suitable for the eating and swallowing function of the subject can be output. In order to enhance the accuracy of evaluation of the eating and swallowing function, machine learning may be used.

Also, for example, if a recipe adjusted to be suitable for the eating and swallowing function in the case where it is determined, based on a feature value, that mastication is not possible is output despite the fact that the subject is capable of mastication without any trouble, the subject can determine that the output recipe is inappropriate. Then, by updating, based on the result of evaluation, various threshold values used to evaluate the eating and swallowing function, an inappropriate recipe as described above is not suggested based on the same feature value. In the manner described above, a recipe based on more accurate evaluation of the eating and swallowing function can be output to the subject. Machine learning may be used to update the threshold value.

Also, for example, the results of the evaluation of the eating and swallowing function may be stored as big data and used in machine learning.

Also, for example, the embodiment given above was described assuming that the subject speaks Japanese, but the subject may speak a language other than Japanese such as English. That is, the speech data that is subjected to signal processing does not necessarily need to be speech data in Japanese, and may be speech data in a language other than Japanese.

Also, for example, the steps included in the recipe output method may be implemented by a computer (computer system). The present invention can be implemented as a program for causing a computer to execute the steps included in the recipe output method. Furthermore, the present invention can be implemented as a non-transitory computer-readable recording medium, such as a CD-ROM, in which the program is recorded.

For example, in the case where the present invention is implemented as a program (software), the steps are executed by the program being executed using hardware resources of the computer such as the CPU, the memory, and the input/output circuit. That is, the steps are executed by the CPU acquiring data from the memory, the input/output circuit, or the like, performing computation, and outputting the result of computation to the memory, the input/output circuit, or the like.

The structural elements included in recipe output system 100 according to the embodiment described above may be implemented as dedicated or general-purpose circuits.

Also, the structural elements included in recipe output system 100 according to the embodiment described above may be implemented as LSI (Large Scale Integration) that is an integrated circuit (IC).

Also, implementation of an integrated circuit is not limited to an LSI, and may be implemented by a dedicated circuit or a general-purpose processor. It is also possible to use an FPGA (Field Programmable Gate Array) that can be programmed or a reconfigurable processor that enables reconfiguration of connection and setting of circuit cells in the LSI.

Furthermore, if a technique for implementing an integrated circuit that can replace LSIs appears by another technique resulting from the progress or derivation of semiconductor technology, integration of the structural elements included in recipe output system 100 may be of course performed by using that technique.

The present invention also encompasses other embodiments obtained by making various modifications that can be conceived by a person having ordinary skill in the art to the above embodiment as well as embodiments implemented by any combination of the structural elements and the functions of the above embodiment without departing from the scope of the present invention.

### REFERENCE SIGNS LIST

24 acquirer
25 outputter
32 input receiver (receiver)
100 recipe output system

1-8. (canceled)

9. A recipe output method comprising:
receiving a selection of a dish from a menu indicating a plurality of dishes;
acquiring ability information indicating an eating and swallowing function of a subject determined by evaluation based on a speech spoken by the subject, the eating and swallowing function including at least two of the following abilities of the subject: biting ability, tongue movement, swallowing action, ability to gather food into a food bolus, movement of jaws, and ability to prevent choking; and
outputting a recipe for cooking the dish for which the selection was received, the recipe being adjusted to be suitable for the eating and swallowing function of the subject indicated by the ability information acquired.

10. The recipe output method according to claim 9, further comprising:
recording the speech spoken by the subject; and
evaluating the eating and swallowing function of the subject based on the speech spoken by the subject recorded and outputting the ability information,
wherein the acquiring the ability information includes acquiring the ability information output.

11. The recipe output method according to claim 9,
wherein the outputting the recipe further includes, when the eating and swallowing function of the subject is less than or equal to a predetermined threshold value, outputting the recipe that includes at least one of the following items suitable for the eating and swallowing function of the subject: a cooking method, an amount of water added, a cutting method for cutting ingredients, a method of preparatory process, and an eating manner.

12. The recipe output method according to claim 9,
wherein the outputting the recipe further includes, when the eating and swallowing function of the subject is less than or equal to a predetermined threshold value, outputting the recipe for cooking the dish for which the selection was received by using ingredients suitable for the eating and swallowing function of the subject.

13. The recipe output method according to claim 9,
wherein the outputting the recipe further includes outputting the recipe adjusted to be suitable for the eating and swallowing function of the subject by making a modification to a standard recipe for cooking the dish for which the selection was received in a normal way by referencing a modification table in which eating and swallowing functions and modifications to be made in the outputting the recipe are associated with each other.

14. The recipe output method according to claim 9,
wherein the outputting the recipe further includes:
when the eating and swallowing function of the subject is less than or equal to a predetermined threshold value, presenting a list indicating a plurality of recommended dishes that are recommended instead of the dish for which the selection was received; and
outputting a recipe for cooking a recommended dish for which a selection from the list indicating the plurality of recommended dishes was received, the recipe being adjusted to be suitable for the eating and swallowing function of the subject indicated by the ability information acquired.

15. A recipe output system comprising:
a receiver that receives a selection of a dish from a menu indicating a plurality of dishes;
an acquirer that acquires ability information indicating an eating and swallowing function of a subject determined by evaluation based on a speech spoken by the subject, the eating and swallowing function including at least two of the following abilities of the subject: biting ability, tongue movement, swallowing action, ability to gather food into a food bolus, movement of jaws, and ability to prevent choking; and
an outputter that outputs a recipe for cooking the dish for which the selection was received, the recipe being adjusted to be suitable for the eating and swallowing function of the subject indicated by the ability information acquired.
